(19) United States
(12) Patent Application Publication
(10) Pub. No.: US 2004/0235048 A1
(43) Pub. Date: Nov. 25, 2004

(54) FUNCTIONALIZED PROTEINACEOUS COATINGS

(76) Inventors: Neville Boden, Leeds (GB); Amalia Agelli, Leeds (GB); Colin Fishwick, Leeds (GB); Charles M. Knobler, Los Angeles, CA (US); Ji Yu Fang, Springfield, VA (US); James Henderson, Leeds (GB)

Correspondence Address: MYERS BIGEL SIBLEY & SAJOVEC, PO BOX 37428, RALEIGH, NC, 27627, US

(21) Appl. No.: 10/474,303

(22) PCT Filed: Apr. 8, 2002
(86) PCT No.: PCT/GB02/01508

(30) Foreign Application Priority Data
Apr. 7, 2001 (GB) 0108767.5

(51) Int. Cl. G01N 33/53; C12M 1/34

(52) U.S. Cl. 435/7.1; 435/287.2

(57) ABSTRACT

There is described a substrate coated with at least a mono layer of a self assembled beta-sheet peptide tape. The mono layer may be substantially flat and have a thickness of ≦5 nm. There is also described a method of coating of a substrate with a peptide mono layer which comprises self-assembly of a peptide monomer into a beta-sheet peptide mono layer on a substrate surface (in situ).

The present invention relates to a novel form of coating, to novel coated substrates, to methods of applying the coating, and to the technological applications of these coatings.

More specifically, the present invention relates to a novel form of molecular coating, especially proteinaceous coatings i.e. peptide coatings and substrates provided with such coatings. The invention also relates to a novel method for deposition of such coatings.

Conventional mono layer coatings are produced by either Langmuir Blodget deposition of surfactant mono layers onto solid substrates or alternatively, by deposition of functionalised thiols onto metatlised (e.g. gold) surfaces or by deposition of polymers onto surfaces.

Currently, biosensor applications utilise protein receptors on a substrate surface by immobilisation of the whole protein molecule receptors. However, such systems are disadvantageous because of, inter alia, the delicate nature and complexity of the protein molecule. Other methods for production of biocompatible surfaces have been explored, these generally take the approach of a covalent attachment of peptide-ligands to a substrate surface, the ligands then act to bind to appropriate cell receptors.

Tirell et al (Langmuir 1997, 13, 4775) describes a pH dependent random coil-to-beta sheet transition of the [(AG)(3)EG](6) peptide at an air-water interface. However, Tirell does not describe the formation of peptide tapes at the interface. Instead Tirell refers generally to the formation of beta-sheet structures. In fact, it is well known that peptides can form β-sheets by intra- or intermolecular hydrogen bonding between two or more β strands.

Sano et al (Langmuir 1999, 15, 513-15) describes mesoscopic tapes formed by amphiphilic two-dimensional colloids on the surface of water. Sano reports the formation of two-dimensional assemblies on a water surface by a short chain carboxyazobenzene derivative. Whilst the resulting structures are “tape-like”, with a thickness of 1 nm, width of 80 nm and 1 nm length, the reported aggregates are not made of a self-assembly of peptide molecules. Therefore the system lacks the capability of functionality and biocompatibility to surfaces designed into it by appropriate peptide choice.

Kowalewski et al, Proc. Natl. Acad. Sci. USA, Vol. 96, pp 3688-3693, has described the aggregation of Alzheimers β-amyloid peptide into linear assemblies reminiscent of protofibrillar species on hydrophobic graphite, some 6-8 nm in diameter.

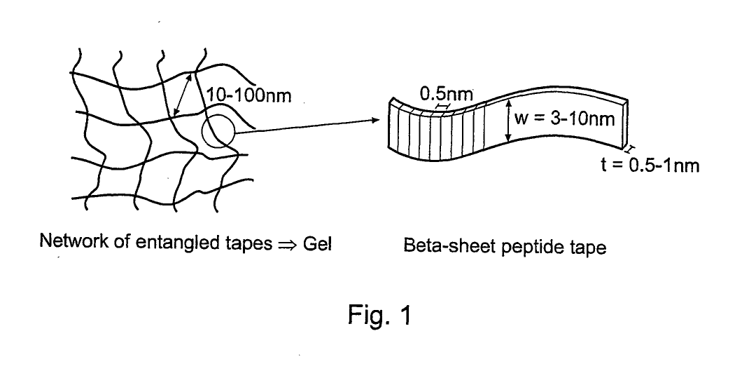

International Patent Application No WO 96/31528, Boden, et al describes novel peptides which self assemble laterally in one dimension to produce beta-sheet tape like polymers. Above a certain critical peptide concentration, the tapes get entangled and form gels under specific solution conditions (FIG. 1). The peptide gels possess the specific property of being able, by external chemical or physical triggering under certain conditions, to switch from the gel state to a fluid or stiffer gel state.

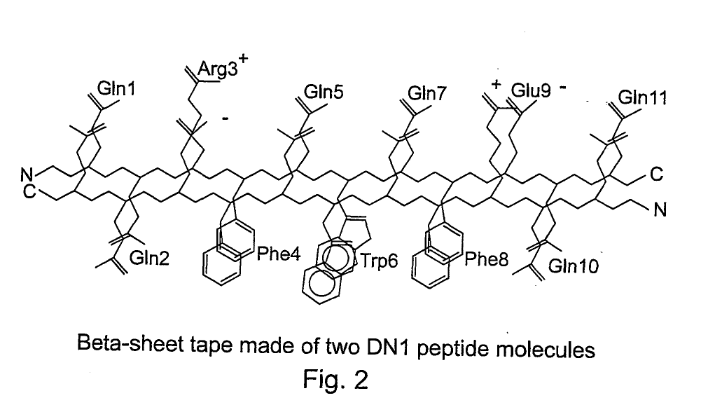

We have now surprisingly found that specific solid substrates can trigger self-assembly of monomeric peptides from the solution, and β-sheet tape formation directly on the substrate. These novel surface coatings based on β-sheet tapes lying flat with one of their sides on the substrate, are advantageous in that, inter alia, they can impart functional group characteristics to that surface by exploiting the periodic structure of a beta-sheet tape (FIG. 2).

Thus according to the invention we provide a substrate coated with at least a mono layer of self assembled beta-sheet peptide tapes.

It is an especially advantageous feature of the invention that the β-sheet tapes may form a monolayer, that is, a layer one molecule in thickness and will be substantially that. However, it is within the scope of this invention to include coated substrates in which the surface coating is more than one molecule thick i.e. it consists of more than one layers of self assembled beta-sheet tapes.

In some applications (eg biosensors) it may be preferable to have surfaces coated with only one well defined layer of tapes. In other applications, it may be preferable to have more than one layer per coating, eg when it is important to produce a stronger coating, for example, for stabilisation of clay surfaces in oil wells.

If only naturally occurring amino acid residues are used, the mono layer may be substantially flat and have a thickness of ≦2 nm. Alternatively, if non-naturally occurring amino acid residues are used the thickness may be dictated by, inter alia, the length of the side chains used and may typically be ≦5 nm.

Since the particular advantage of the beta-sheet peptide tapes is their ability to form a mono layer, we especially provide a substrate as hereinbefore described wherein the beta-sheet peptide tape mono layer has a thickness of 2 mn or less.

We especially provide a substrate as hereinbefore described wherein one side of the beta-sheet peptide tape has substantially high affinity for the substrate surface. In particular, a first side of the beta-sheet peptide tape has a higher affinity for the substrate surface than a second side of the tape.

The peptides that form the surface coatings may be at least 3 amino-acids long, that is, have 3 amino acid residues, e.g. their length may vary from 3 to 30 residues.

The peptide mono layer coating may be made responsive to external triggers, e.g. to pH changes. This is possible because, inter alia, the peptides are not covalently attached on the substrate surface and therefore their surface coatings may provide improved responsiveness over the thiol technology of the prior art.

The peptide mono layer coating method provides a simple, well-controlled and reproducible way of designing functional receptor sites on a substrate surface for, e.g. biosensor, separation applications and cell interactions, by utilising the specific geometric arrangement of the amino acid side chains on the beta-sheet scaffold.

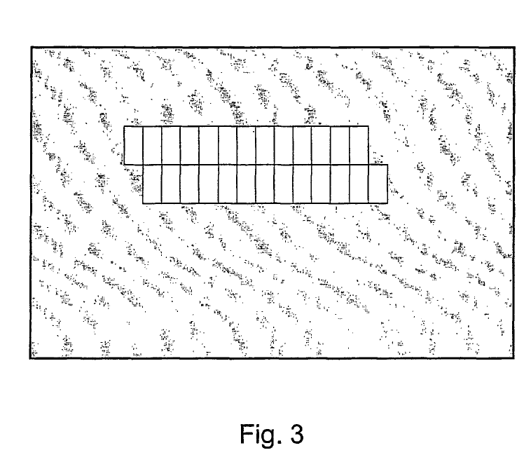

The robustness of the coating depends to a lesser extent on the edge-to-edge interactions between tapes (FIG. 3) and to a greater extent on the strength of the β-tape-substrate interaction energy, eg β-tapes interacting via their hydrophobic side with a hydrophobic substrate will create a robust coating if the substrate is immersed in a polar solvent, eg water.

The peptide coating can be rendered even more permanent by inducing covalent bonding between the peptide coating and the substrate, as well as between peptide molecules, after the initial formation of the coating on the substrate.

These self assembling beta-sheet peptide mono layer coatings on substrate surfaces can impart specific functionality on the surface, including biocompatibility and bioresponsivity, and specific recognition properties. The coatings are also environmentally non-toxic.

The affinity which one side of a beta-sheet peptide tape has for a substrate surface can originate from a number of sources, for example, from coulombic attractions between the peptide tape and the substrate surface, alternatively, it can be based on hydrophobic interactions or hydrogen bonding, or a combination of any of these factors. The side of the tape which faces up, i.e. it is remote from the substrate surface, preferably has a lower affinity for the substrate surface (which is referred to as the low affinity side of the tape).

Tapes with chemically distinct sides such as described above, can be formed by peptides whose primary structure is, at least in part, an alternating one: X Y X Y etc,

wherein X is an amino acid residue with a side chain possessing high affinity for the substrate surface; and

Y is an amino acid residue with a side chain possessing low affinity for the substrate surface.

A peptide with a partial XYXY etc motif in its primary structure is illustrated in FIG. 2. Indeed such tapes on a substrate surface, with a substantially repeating alternating XYXY primary structure are also novel per se.

Thus according to a further feature of the invention we provide a substrate coated with a layer of flat self assembled beta-sheet peptide tapes which possess a substantially repeating alternating peptide primary structure, e.g. of the kind XYXY.

In an especially preferred embodiment the edges of a first tape have a high affinity for the edges of a second tape, i.e. the tapes possess edge-to-edge interactions. Such favourable edge-to-edge interactions may provide lateral association between tapes and thus good coverage of a substrate surface by the peptide mono layer and also a more robust coating. Such interactions may be built into the primary peptide structure, e.g. by the presence of complementary chemical groups at the N- and C-termini of the peptide chain. Such complementary chemical groups can be for example:

1. Complementary hydrogen bonding groups, such as an acetylated N-terminus, e.g. CH₃CONH— and amidated C-terminus, e.g. —CONH₂; this is the case for example for DN1 peptide as the primary structure.

2. Complementary coulombic interactions, for example, a positive charge at the N-terminus, e.g. —NH₃⁺ and a negative charge at the C-terminus, e.g. —COO⁻.

3. Hydrophobic interactions.

In a further preferred embodiment of the invention the peptides may be chemically cross-linked to each other and/or to the substrate, such cross-linking may enhance the long term stability of the mono layer.

In additions the peptides in the mono layer may be self-assembled, e.g. on a patterned surface, to produce coating with a greater degree of functionality. In this way, surfaces can be produced which are partially covered with a peptide coating only at desirable places. Or there is also the opportunity to produce surfaces with mixed peptide coatings. The coatings will be formed on different parts of the surface guided by the surface pattern.

The primary peptide structures may be selected such that the peptide molecules can interact specifically with each other and self-assemble in one dimension e.g. to form infinitely long polymeric beta-sheet tapes.

When the peptide mono layer is no longer required the mono layer may be removed by, e.g. chemical triggers. The choice of such chemical triggers will vary and will depend upon, inter alia, the peptide(s) in the peptide mono layer, other methods may also be used.

The peptide coating on the solid surface can be produced using a variety of different methods. Such methods may include but shall not be limited to the following:

(i) immersion of a substrate in a peptide solution (where a significant fraction of the peptide molecules is in a monomeric state), followed by spontaneous self assembly of the peptide tapes on the surface of the substrate;

(ii) spin coating, which may comprise depositing a peptide solution on a spinning substrate and allowing it to evaporate causing spontaneous self assembly of the peptides;

(iii) Langmuir Blodgett deposition of preformed tapes, eg monolayer tapes, from an air/solvent interface; and

(iv) solvent evaporation, in which a substrate is brought into contact with a peptide solution, the substrate is subsequently aired and upon evaporation of the solvent the peptides self assemble.

If the substrate has an alternating or periodic structure, it can act as a template for peptide arrangement on the substrate surface, for example, tapes formed on a mica surface may have a hexagonal arrangement on the surface imposed by the hexagonal mica lattice structure.

Examples of substrates include, but are not limited to, SAMS (self assembly monolayers), eg thiols, glass; mica; polymeric surfaces; metals, such as AU; etc.

According to a further feature of the invention we provide the use of a peptide in the manufacture of a peptide monolayer as hereinbefore described.

We especially provide the use of a β-sheet peptide tape in the manufacture of a coated substrate as hereinbefore described.

The small size of the initially monomeric peptide allows it to be transported even inside very small cavities or through very narrow capillaries to the deposition site.

Furthermore, the peptide(s) may be easily transported in the monomer state to the site of interest where they can be triggered to self-assemble into a mono layer to cover the required solid substrate.

Thus, we also provide a method of coating of a substrate with a peptide mono layer as hereinbefore described which comprises self-assembly of a peptide monomer into a β-sheet peptide layer on a substrate surface in situ.

The peptides used In the mono layer can be designed to be biocompatible and biodegradable.

The method of the invention is advantageous because, inter alia, production of the peptide coatings is very simple, quick and cost effective, since a significant modification in surface properties can be achieved with very small amount of material.

The methodology of the invention may be applied to any surface by appropriate modification of the peptide design. In contrast, techniques which employ covalent attachment work only with specifically tailored substrates. The peptide monolayers are produced using intrinsic self-assembly methods.

The physicochemical properties of the surface may be controlled by design of the self-assembly peptides. For example the thickness or width of the tapes, their responsiveness to external triggers, and/or the chemical nature of either side or both sides of the tapes can be controlled by peptide design.

The self assembled peptides are biocompatible and environmentally friendly. The peptides themselves are chemically stable up to 350° C. etc.

The materials can be produced in large scale and inexpensively by biotechnology routes or by transgenic plants or animals.

The novel coatings are advantageous in that, inter alia, they have potential applications in the following areas:

Industrial applications and new products:

Modification of the physical and chemical properties of a surface in a controlled way, e.g. wetting properties; for example, for anti-icing applications. Also for controlling the interaction of oil/water with clay surfaces, and the stabilising the clay itself, an important issue when e.g. dealing with fractures in oil-wells.

Receptor sites can be engineered by design into the surface coating, providing materials for use as sensors or as biocatalysts, or as separation media in biotechnology applications.

The peptide coatings can be engineered to control the chemical and bioactive properties of synthetic polymer fibres. In this application, the peptides are envisaged to self-assemble directly on the surface of the synthetic polymer fibres. This methodology has the advantage of harnessing and combining existing expertise on manufacturing at low-cost well controlled fibrous structures with desirable mechanical properties, with the opportunity of designing their bioactivity, biocompatibility and other chemical properties by coating with the peptide mono layers.

Adhesives (e.g. molecular Velcro™); for example, mono layers with two chemically distinct sides, e.g. polar and a polar surfaces, can be used to bring two chemically incompatible surfaces together.

Biomedical and biomaterial applications, such as:

Biomineralisation using the peptide mono layer materials as a template for the nucleation and growth of inorganic materials.

Bioresponsive and biocompatible surfaces produced by adhesion, spreading and growth of endothelial cells on the surface of medical implant materials.

Artificial skin.

Tissue adhesives.

The tapes can be used to provide paths/tracks, to control and guide the direction of growth or movement of molecules or cells. This may be significantly useful for nerve tissue repair as well as for growth and formation of bone tissue (tissue engineering).

Personal care products, such as:

Dental applications: peptide coatings for protection of teeth and for nucleation of hydroxyapatite.

Skin treatment products.

The peptide coatings can also be produced by the mixing of more than one type of peptide molecules, eg peptides A and B. If A and B peptides have complementary primary structures, they will self-assemble into heteropolymeric beta-sheet tapes ABAB . . . on the surface. This approach allows to extend the range of chemical properties and periodic features that can be engineered on the peptide monolayer on the surface, and is particularly useful for extending the range of molecular recognition sites that can be designed on the surface.

If the tape/substrate interactions are strong, then a good coating is formed even when the substrate is still immersed in the peptide solution. If the tape/substrate interactions are weak, then the coating may be formed when the peptide solution is aired on the substrate surface. Furthermore, the extent of coverage depends upon the strength of both tape/substrate interactions and also tape/tape interactions; and also on the concentration of the peptide monomer in the solution.

The preferred method of manufacture comprises bringing the solution of monomeric peptide into contact with the surface of interest. The solution is either left to dry on this surface, or the surface is immersed into this solution for some time (typically 30-60 minutes, but this can vary). During either of these processes a carpet/coating of flat self-assembled peptide tapes is produced on the surface

Also, monolayers may be formed at an air-water interface and then transferred onto a substrate.

The invention will now be described by way of Example only.

### EXAMPLES

1.1 Method I

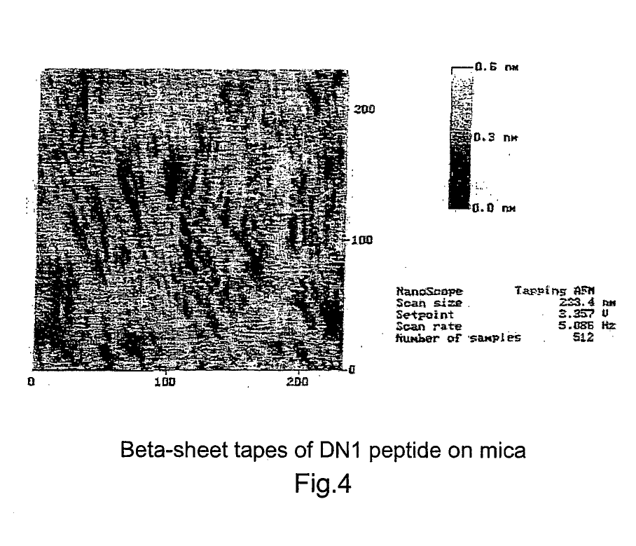

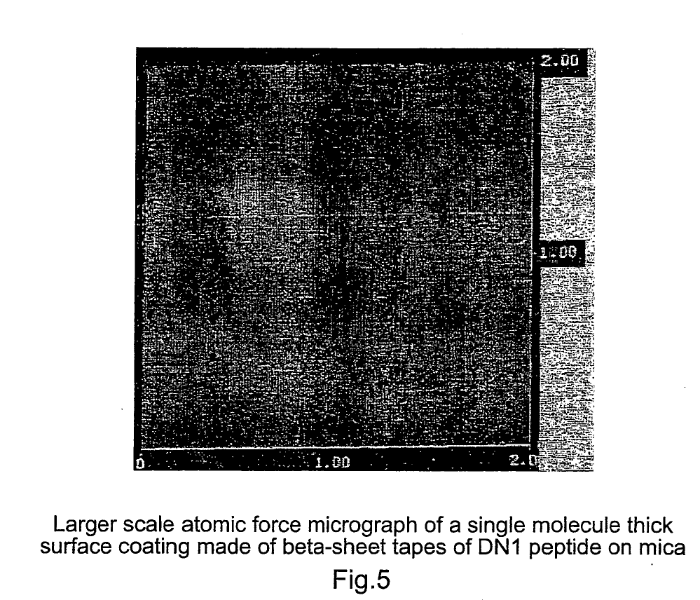

DN1, at low concentration in water (typically <30 μM, QQRFQWQFEQQ, has been shown to be a random coil monomer by circular dichroism and time resolved fluorescence anisotropy studies. 10 μL droplets of an aqueous solution of DN1 monomers (10 μM DN1 in water) were deposited on freshly cleaved, atomically flat mica, the surface of the solution was left to dry and then imaged by AFM (FIGS. 4 and 5).

Following the formation of the peptide coating, the mica surface becomes significantly more hydrophobic compared to bare mica. This is demonstrated by the fact that water spreads much less on mica which has the peptide coating, compared to bare mica. This leads us to propose that DN1 tapes interact with the mica via their polar side (upper side in FIG. 2) through electrostatic forces, whilst they expose their other more hydrophobic side (lower side of tapes in FIG. 2) to the air.

The self-assembly of the peptide tape coating can alternatively take place on surfaces with a prepatterned variation of surface energies, so that the monolayer is only formed on specific patches on the surface. Spin-coating will also be employed for the deposition of uniform thickness self-assembled monolayer on the surface.

By blotting the excess solution off the surface, partial orientation of the tapes on the surface is achieved (molecular combing). The use of magnetic or electric fields or/and oriented grooves on the surface can also be used to control the alignment of the tapes on the surface.

1.2 Method II

Freshly cleaved mica was immersed in dilute monomeric DN1 solution in water for increasing amount of time. Subsequently, it was removed, dried naturally at ambient temperature, and imaged by AFM operating in contact or tapping mode. The longer the mica was immersed in the monomeric peptide solution for, the more peptide molecules had the chance to interact with the mica surface, and consequently, the higher the concentration of the self-assembled peptide tapes formed on it. This observed growth of density of the peptidic tapes on the mica surface in solution, over time, together with the close structural similarities of the tapes prepared in the two different ways, indicate that these polymers are not formed in solution due to increased peptide concentration during water evaporation.

The monolayer once formed can then be chemically crosslinked to become permanent.

During either of these processes, a monolayer coating comprised of tape aggregates arranged side-by-side is produced.

1.3 Method III

An alternative method of self-assembly of the peptide monolayers is at an air-water interface by the Langmuir-Blodget dipping method and subsequent deposition onto a solid substrate.

Peptides appropriate for method III may be designed to be insoluble in water. In contrast, peptides appropriate for Methods I and II would be designed to be soluble in water or appropriate polar organic solvents.

Figure Legends

FIG. 1

Schematic representation of peptides in beta-strand conformation (represented as vertical lines) hydrogen bonding in one dimension with each other to form long self-assembling beta-sheet tapes. The width of the tape is determined by the length of the peptide molecules. The thickness of the tape is equal to the thickness of a beta-strand. The surface properties of the tapes are defined by the end groups of the peptide amino acid side-chains. The tapes are also shown to entangle to form a gel network in a good solvent.

FIG. 2:

Representation of a short segment of a β-sheet tape made of the self-assembly of two antiparallel 11-residue peptides in β-strand conformation. The actual peptide primary structure depicted in this figure belongs to the DN1 peptide (QQRFQWQFEQQ). The peptides interact in such a way that the polar sides of both peptides face up, whilst the a polar sides of both peptides face down. Thus the growing β-sheet tape will have two distinct sides: a polar and an a polar one, determined by the amino-acid side-chains. The β-sheet tape is stabilised via intermolecular peptide backbone hydrogen bonds and a plurality of amino-acid side-chain interactions. The distance between main long axes of adjacent peptides in a β-tape is 0.47-0.50 nm. The distance between alternating amino-acid side-chains (e.g. at positions 1 and 3 along the peptide β-strand) is around 0.66-0.7 nm.

FIG. 3:

Drawing showing a surface coating made of flat β-sheet tapes (each shown as a ladder) of self-assembling peptides (each peptide chain is shown by a short straight line) on a solid substrate. The tapes interact edge-to-edge (i.e. laterally) with each other.

FIG. 4:

An atomic force micrograph of flat tapes of the 11-residue DN1 peptide self-assembled directly on mica surface from a monomeric peptide solution in water in contact with the mica The width of the tapes is ca 4 nm (consistent with the expected length of an 11-residue peptide). Each tape is hundreds of nanometers long. The thickness of the tapes is around 0.7 nm, consistent with the expected thickness of a single-molecule thick β-sheet.

FIG. 5:

Atomic force micrograph of a larger area of the mica showing that most of the surface area of the substrate is covered by flat tapes packed laterally against each other, forming a thin uniform film coverage.

1. A solid substrate coated with at least a mono layer of a self assembled beta-sheet peptide tape wherein the solid substrate, when in contact with a solution of monomeric peptide, can trigger self assembly of monomeric peptides to form a beta-sheet tape peptide.

2. The substrate according to claim 1 characterized in that the layer is a monolayer.

3. The substrate according to claim 1 characterized in that one side of the beta-sheet peptide tape has a substantially high affinity for the substrate surface.

4. The substrate according to claim 3 characterized in that a second side of the tape having functional receptor sites on the surface is provided.

5. The substrate according to claim 1 characterized in that the tape is environmentally non-toxic.

6. The substrate according to claim 3 characterized in that the affinity which the tape has for the substrate originates from coulombic attractions between the peptide tape and the surface.

7. The substrate according to claim 3 characterized in that the affinity which the tape has for the substrate originates from hydrophobic interactions.

8. The substrate according to claim 3 characterized in that the affinity which the tape has for the substrate originates from hydrogen bonding.

9. The substrate according to claim 3 characterized in that the other side of the tape has a lower affinity for the substrate surface.

10. The substrate according to claim 1 characterized in that tape is formed from peptides having a primary structure comprising a substantially repeating alternating structure.

11. The substrate according to claim 1 characterized in that the edges of the tape have high affinity for the edges of an adjacent tape.

12. The substrate according to claim 11 characterized in that such interactions are based on the presence of complementary chemical groups at the N- and C-termini of the peptide chain.

13. The substrate according to claim 11 characterized in that the interactions are complementary hydrogen bonding groups.

14. The substrate according to claim 1 characterized in that the interactions are from coulombic interactions.

15. The substrate according to claim 1 characterized in that the interactions are hydrophobic interactions.

16. The substrate according to claim 1 characterized in that the peptides are chemically cross-linked to each other to enhance the long term stability of the mono layer.

17. The substrate according to claim 1 characterized in that the mono layer is self-assembled.

18. The substrate according to claim 1 characterized in that the peptide primary structures are designed such that peptide molecules can interact with each other and self-assemble in one dimension to form infinitely long polymeric beta-sheet tapes.

19. The substrate according to claim 1 characterized in that the peptide coating mono layer is made responsive to pH changes.

20. The substrate according to claim 1 characterized in that the monolayer is one molecule in thickness.

21. The substrate according to claim 1 characterized in that the beta-sheet peptide tape has a thickness of 2 nm or less.

22. The substrate according to claim 1 characterized in that the peptide is modified to have anti-icing applications.

23. The substrate according to claim 1 characterized in that the peptide is modified to control the interaction of oil/water with clay surfaces.

24. The substrate according to claim 1 characterized in that the peptide is modified to control the chemical and/or bioactive properties of a synthetic polymer fiber.

25. The substrate according to claim 1 characterized in that the peptide is modified as a template for the nucleation and growth of inorganic materials.

26. The substrate according to claim 1 characterized in that the peptide is modified to produce a bioresponsive or biocompatible surface, or combinations thereof, produced by adhesion, spreading and growth of endothelial cells on the surface of medical implant materials.

27. The substrate according to claim 1 characterized in that the peptide is modified to produce an artificial skin.

28. The substrate according to claim 1 characterized in that the peptide is modified to produce a tissue adhesive.

29. The substrate according to claim 1 characterized in that the peptide is modified to produce a biocompatible surface for nerve tissue repair, bone tissue formation or tissue engineering, or combinations thereof.

30. The substrate according to claim 1 characterized in that the peptide is modified to produce a material as a template for the nucleation or growth, or combinations thereof, of inorganic materials.

31. The substrate according to claim 1 characterized in that the peptide is modified to control the chemical or bioactive properties, or combinations thereof, of synthetic polymer fibers.

32. A substrate according to claim 1 characterized in that the peptide is modified to provide a peptide coating for protection of teeth or nucleation of hydroxyapatite, or combinations thereof.

33. A substrate according to claim 1 characterized in that the peptide is modified to provide a skin treatment product.

34. The method of using a peptide in the manufacture of a β-sheet peptide tape mono layer.

35. The method of using β-sheet peptide tape in the manufacture of a substrate according to claim 1.

36. A method of coating a substrate with a peptide mono layer which comprises self-assembly of a peptide monomer into a beta-sheet peptide mono layer on a substrate surface in situ.

37. A method of simultaneous self-assembly on a substrate surface of a beta-sheet tape monolayer which comprises more than one type of peptide molecule, to produce a heteropolymeric beta-sheet tapes ABAB . . . on the substrate surface.

38. The method according to claim 36 which comprises-bringing a solution of a monomeric peptide into contact with a substrate surface, to dry on this surface, or the surface is immersed into this solution and then left to dry to produce a carpet/coating or self-assembled peptide tapes lining flat on the substrate surface.

39. A substrate or a method substantially as described with reference to the accompanying examples.
